(19) United States
(12) Patent Application Publication
(10) Pub. No.: US 2025/0244574 A1
(43) Pub. Date: Jul. 31, 2025

(54) Hybrid Gaze Tracking Circuitry

(71) Applicant: Apple Inc., Cupertino, CA (US)

(72) Inventor: Patrick R Gill, Victoria (CA)

(21) Appl. No.: 18/975,954

(22) Filed: Dec. 10, 2024

Related U.S. Application Data
(60) Provisional application No. 63/626,390, filed on Jan. 29, 2024.

(51) Int. Cl. G02B 27/00; G02B 27/01

(52) U.S. Cl. CPC G02B 27/0093; G02B 27/0172; G02B 2027/0134; G02B 2027/0138; G02B 2027/0178; G02B 2027/0187

(57) ABSTRACT

Eyewear such as a head-mounted device may include adjustable prescription lenses and/or may include displays. The eyewear may include gaze tracking circuitry that tracks a gaze direction of a user. The gaze tracking circuitry may include a camera, a light source, and a range finder. The light source may be a light-emitting diode that produces an eye glint on the user's eye. The camera may capture an eye image including an image of the glint. Pupil location and glint location may be determined based on the captured image. The range finder may measure an eye distance to the user's eye. Gaze direction may be determined based on the pupil location, the glint location, and the eye distance, even when only one eye glint is captured by the camera. The range finder may be an ultrasonic range finder, an optical range finder, or any other suitable range finder.

This application claims the benefit of U.S. provisional patent application No. 63/626,390, filed Jan. 29, 2024, which is hereby incorporated by reference herein in its entirety.

### FIELD

This relates generally to electronic devices, and, more particularly, to wearable electronic devices such as head-mounted devices.

### BACKGROUND

Head-mounted devices and other eyewear may use gaze tracking circuitry to track a user's gaze.

It can be challenging to design gaze tracking circuitry that performs satisfactorily. If care is not taken, the gaze tracking circuitry may produce inaccurate measurements or may exhibit other performance limitations such as excessive power consumption.

### SUMMARY

Eyewear such as a head-mounted device may include adjustable prescription lenses and/or may include displays. The lenses and displays may be mounted to a support structure such as supporting frames or other head-mounted support structures.

The eyewear may include gaze tracking circuitry that tracks a gaze direction of a user. The gaze tracking circuitry may include a camera, a light source, and a range finder. The light source may be a light-emitting diode that produces an eye glint on the user's eye. The camera may capture an eye image including an image of the glint. Pupil location and glint location may be determined based on the captured image.

The range finder may measure an eye distance to the user's eye. Gaze direction may be determined based on the pupil location, the glint location, and the eye distance, even when only a single eye glint is captured by the camera. The range finder may be an ultrasonic range finder, an optical range finder, or any other suitable range finder.

If desired, the camera and light source may be maintained in a low-power state until the range finder detects a change in eye distance (which may indicate a change in gaze direction). In response to detecting the change in eye distance, the light source and camera may be turned on to measure pupil location and glint location so that a new gaze direction can be determined based on the new eye distance.

### BRIEF DESCRIPTION OF THE DRAWINGS

FIG. 1 is a top view of an illustrative head-mounted device that may include gaze tracking circuitry in accordance with an embodiment.

FIG. 2 is a rear view of an illustrative head-mounted device that may include gaze tracking circuitry in accordance with an embodiment.

FIG. 3 is a schematic diagram of an illustrative head-mounted device that may include gaze tracking circuitry in accordance with an embodiment.

FIG. 4 is a top view of illustrative gaze tracking circuitry being used to track a gaze direction of a user in accordance with an embodiment.

FIG. 5 is a side view of a portion of an illustrative head-mounted device having an optical module that includes a display, a lens, and gaze tracking circuitry in accordance with an embodiment.

FIG. 6 is a top view of an illustrative range finder that includes an array of ultrasonic transducers in accordance with an embodiment.

FIG. 7 is a side view of an illustrative range finder that includes a self-mixing interferometer in accordance with an embodiment.

FIG. 8 is a graph showing an illustrative quantitative model for determining gaze direction using a range finder, a camera, and a light source in accordance with an embodiment.

FIG. 9 is a flow chart of illustrative steps involved in tracking gaze direction using a range finder, a camera, and a light source in accordance with an embodiment.

### DETAILED DESCRIPTION

Eyewear such as a pair of glasses or other head-mounted device may include one or more eye monitoring components such as gaze tracking circuitry. These components may include, for example, one or more cameras, one or more light sources, and one or more range finders (e.g., distance sensors). The light source may illuminate the user's eye while the camera captures an image of the eye. In an illustrative configuration, the light source may include a light-emitting diode that creates a glint on the user's eye and/or that illuminates the user's pupil and iris. Pupil location and glint location may be determined based on the eye images captured by the camera. The range finder may include an optical range finder, an ultrasonic range finder, or any other suitable range finder and may be used to determine a distance to the eye (sometimes referred to as eye distance). The gaze direction of the user may be determined based on the location of the glint, the location of the pupil, and the distance to the eye.

Using hybrid gaze tracking circuitry that includes both a camera and light source for glint detection and a range finder for distance sensing may allow gaze direction to be determined using fewer glints (e.g., using only a single glint from a single light-emitting diode, if desired). This may be beneficial in arrangements where fewer light sources are desired and/or in scenarios where multiple glints cannot be obtained from a particular user's eye. In some arrangements, the camera, light source, and range finder may remain powered on during operation of device 10 and may continuously or periodically be used to track gaze direction. In other arrangements, power savings may be achieved by keeping the camera and/or the light source off (or otherwise in a low-power state) until the range finder detects a change in eye distance, which may be indicative of a change in gaze direction. Upon detecting the change in eye distance with the range finder, the camera and light source may be switched on to help determine the new gaze direction of the user.

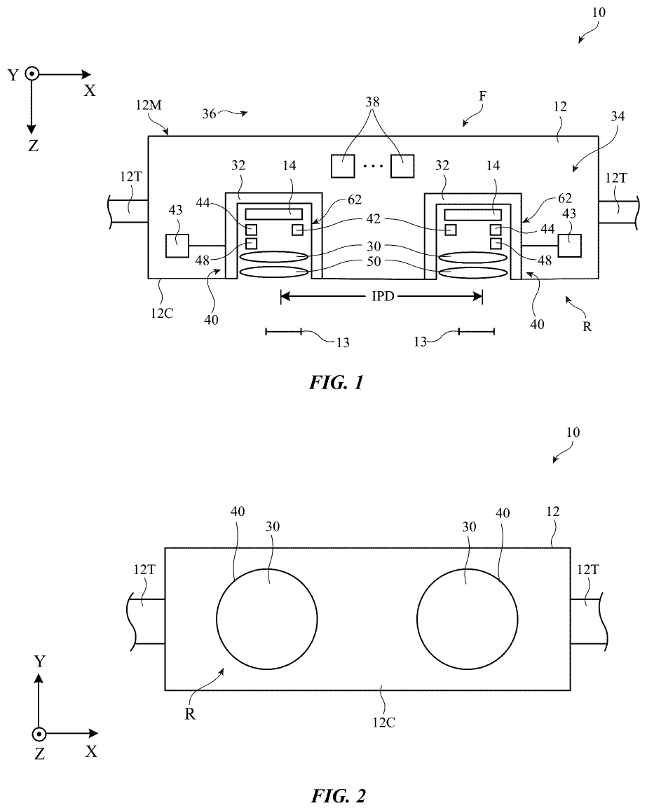

A top view of an illustrative head-mounted device or other eyewear is shown in FIG. 1. As shown in FIG. 1, head-mounted devices such as electronic device 10 may have head-mounted support structures such as housing 12. Housing 12 may include portions (e.g., support structures 12T) to allow device 10 to be worn on a user's head. Support structures 12T may be formed from fabric, polymer, metal, and/or other material. Support structures 12T may form a strap or other head-mounted support structures to help support device 10 on a user's head. A main support structure (e.g., main housing portion 12M) of housing 12 may support electronic components such as displays 14. Main housing portion 12M may include housing structures formed from metal, polymer, glass, ceramic, and/or other material. For example, housing portion 12M may have housing walls on front face F and housing walls on adjacent top, bottom, left, and right side faces that are formed from rigid polymer or other rigid support structures and these rigid walls may optionally be covered with electrical components, fabric, leather, or other soft materials, etc. The walls of housing portion 12M may enclose internal components 38 in interior region 34 of device 10 and may separate interior region 34 from the environment surrounding device 10 (exterior region 36). Internal components 38 may include integrated circuits, actuators, batteries, sensors, and/or other circuits and structures for device 10. Housing 12 may be configured to be worn on a head of a user and may form glasses, a hat, a helmet, goggles, and/or other head-mounted device. Configurations in which housing 12 forms goggles may sometimes be described herein as an example.

Front face F of housing 12 may face outwardly away from a user's head and face. Opposing rear face R of housing 12 may face the user. Portions of housing 12 (e.g., portions of main housing 12M) on rear face R may form a cover such as cover 12C (sometimes referred to as a curtain). The presence of cover 12C on rear face R may help hide internal housing structures, internal components 38, and other structures in interior region 34 from view by a user.

Device 10 may have left and right optical modules 40. Each optical module may include a respective display 14, lens 30, and support structure 32. Support structures 32, which may sometimes be referred to as lens barrels or optical module support structures, may include hollow cylindrical structures with open ends or other supporting structures to house displays 14 and lenses 30. Support structures 32 may, for example, include a left lens barrel that supports a left display 14 and left lens 30 and a right lens barrel that supports a right display 14 and right lens 30.

Displays 14 may include arrays of pixels or other display devices to produce images. Displays 14 may, for example, include organic light-emitting diode pixels formed on substrates with thin-film circuitry and/or formed on semiconductor substrates, pixels formed from crystalline semiconductor dies, liquid crystal display pixels, scanning display devices, and/or other display devices for producing images.

Lenses 30 may include one or more lens elements for providing image light from displays 14 to respective eyes boxes 13. Lenses 30 may be implemented using refractive glass lens elements, using mirror lens structures (catadioptric lenses), using Fresnel lenses, using holographic lenses, and/or other lens systems.

When a user's eyes are located in eye boxes 13, displays (display panels) 14 operate together to form a display for device 10 (e.g., the images provided by respective left and right optical modules 40 may be viewed by the user's eyes in eye boxes 13 so that a stereoscopic image is created for the user). The left image from the left optical module fuses with the right image from a right optical module while the display is viewed by the user.

If desired, device 10 may include additional lenses such as lenses 50. Lenses 50 may be fixed lenses or may be adjustable lenses such as liquid crystal lenses, fluid-filled lenses, or other suitable adjustable lenses. Lenses 50 may be configured to accommodate different focal ranges and/or to correct for vision defects such as myopia, hyperopia, presbyopia, astigmatism, higher-order aberrations, and/or other vision defects. For example, lenses 50 may be adjustable prescription lenses having a first set of optical characteristics for a first user with a first prescription and a second set of optical characteristics for a second user with a second prescription. Lenses 50 may be removably or permanently attached to housing 12. In arrangements where lenses 50 are removable, lenses 50 may have mating engagement features, magnets, clips, or other attachment structures that allow lenses 50 to be attached to housing 12 (e.g., individually or as a pair).

If desired, device 10 may be used purely for vision correction (e.g., device 10 may be a pair of spectacles, glasses, etc.) and some of the other components in FIG. 1 such as displays 14, lenses 30, and optical modules 40 may be omitted. In other arrangements, device 10 (sometimes referred to as eyewear 10, glasses 10, head-mounted device 10, etc.) may include displays that display virtual reality, mixed reality, and/or augmented reality content. With this type of arrangement, lenses 50 may be prescription lenses and/or may be used to move content between focal planes from the perspective of the user. If desired, lenses 50 may be omitted.

Arrangements in which device 10 is a head-mounted device with one or more displays are sometimes described herein as an illustrative example.

It may be desirable to monitor the user's eyes while the user's eyes are located in eye boxes 13. For example, it may be desirable to use a camera to capture images of the user's irises (or other portions of the user's eyes) for user authentication. It may also be desirable to monitor the direction of the user's gaze. Gaze tracking information may be used as a form of user input and/or may be used to determine where, within an image, image content resolution should be locally enhanced in a foveated imaging system. To ensure that device 10 can capture satisfactory eye images while a user's eyes are located in eye boxes 13, each optical module 40 may be provided with gaze tracking circuitry 62. Gaze tracking circuitry 62 may include one or more cameras such as camera 42, one or more light sources such as light source 44 (e.g., light-emitting diodes, lasers, lamps, etc.), and one or more range finders such as range finder 48.

Cameras 42 and light-emitting diodes 44 may operate at any suitable wavelengths (visible, infrared, and/or ultraviolet). With an illustrative configuration, which may sometimes be described herein as an example, diodes 44 emit infrared light that is invisible (or nearly invisible) to the user. This allows eye monitoring operations to be performed continuously without interfering with the user's ability to view images on displays 14.

Range finder 48 (sometimes referred to as depth sensor 48) may be any suitable range finder such as an optical range finder (e.g., a light source and light sensor that gather time-of-flight measurements, phase-based measurements, self-mixing sensors, light detection and ranging (lidar) sensors, structured light sensors, and/or depth sensors based on stereo imaging devices that capture three-dimensional images, etc.), an ultrasonic range finder (e.g., one or more capacitive micromachined ultrasonic transducers, piezoelectric micromachined transducers, and/or other suitable ultrasonic transducers for emitting and/or detecting acoustic signals), and/or any other suitable range finder.

Not all users have the same interpupillary distance IPD. To provide device 10 with the ability to adjust the interpupillary spacing between modules 40 along lateral dimension X and thereby adjust the spacing IPD between eye boxes 13 to accommodate different user interpupillary distances, device 10 may be provided with actuators 43. Actuators 43 can be manually controlled and/or computer-controlled actuators (e.g., computer-controlled motors) for moving support structures 32 relative to each other. Information on the locations of the user's eyes may be gathered using, for example, cameras 42. The locations of eye boxes 13 can then be adjusted accordingly.

As shown in FIG. 2, cover 12C may cover rear face F while leaving lenses 30 of optical modules 40 uncovered (e.g., cover 12C may have openings that are aligned with and receive modules 40). As modules 40 are moved relative to each other along dimension X to accommodate different interpupillary distances for different users, modules 40 move relative to fixed housing structures such as the walls of main portion 12M and move relative to each other.

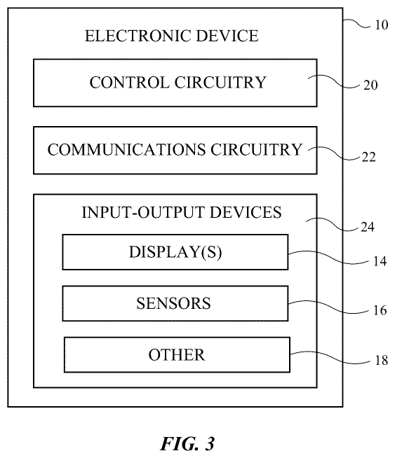

A schematic diagram of an illustrative electronic device such as a head-mounted device or other wearable device is shown in FIG. 3. Device 10 of FIG. 3 may be operated as a stand-alone device and/or the resources of device 10 may be used to communicate with external electronic equipment. As an example, communications circuitry in device 10 may be used to transmit user input information, sensor information, and/or other information to external electronic devices (e.g., wirelessly or via wired connections). Each of these external devices may include components of the type shown by device 10 of FIG. 3.

As shown in FIG. 3, a head-mounted device such as device 10 may include control circuitry 20. Control circuitry 20 may include storage and processing circuitry for supporting the operation of device 10. The storage and processing circuitry may include storage such as nonvolatile memory (e.g., flash memory or other electrically-programmable-read-only memory configured to form a solid state drive), volatile memory (e.g., static or dynamic random-access-memory), etc. Processing circuitry in control circuitry 20 may be used to gather input from sensors and other input devices and may be used to control output devices. The processing circuitry may be based on one or more microprocessors, microcontrollers, digital signal processors, baseband processors and other wireless communications circuits, power management units, audio chips, application specific integrated circuits, etc. During operation, control circuitry 20 may use display(s) 14 and other output devices in providing a user with visual output and other output.

To support communications between device 10 and external equipment, control circuitry 20 may communicate using communications circuitry 22. Circuitry 22 may include antennas, radio-frequency transceiver circuitry, and other wireless communications circuitry and/or wired communications circuitry. Circuitry 22, which may sometimes be referred to as control circuitry and/or control and communications circuitry, may support bidirectional wireless communications between device 10 and external equipment (e.g., a companion device such as a computer, cellular telephone, or other electronic device, an accessory such as a point device, computer stylus, or other input device, speakers or other output devices, etc.) over a wireless link. For example, circuitry 22 may include radio-frequency transceiver circuitry such as wireless local area network transceiver circuitry configured to support communications over a wireless local area network link, near-field communications transceiver circuitry configured to support communications over a near-field communications link, cellular telephone transceiver circuitry configured to support communications over a cellular telephone link, or transceiver circuitry configured to support communications over any other suitable wired or wireless communications link. Wireless communications may, for example, be supported over a Bluetooth® link, a WiFi® link, a wireless link operating at a frequency between 10 GHz and 400 GHz, a 60 GHz link, or other millimeter wave link, a cellular telephone link, or other wireless communications link. Device 10 may, if desired, include power circuits for transmitting and/or receiving wired and/or wireless power and may include batteries or other energy storage devices. For example, device 10 may include a coil and rectifier to receive wireless power that is provided to circuitry in device 10.

Device 10 may include input-output devices such as devices 24. Input-output devices 24 may be used in gathering user input, in gathering information on the environment surrounding the user, and/or in providing a user with output. Devices 24 may include one or more displays such as display(s) 14. Display(s) 14 may include one or more display devices such as organic light-emitting diode display panels (panels with organic light-emitting diode pixels formed on polymer substrates or silicon substrates that contain pixel control circuitry), liquid crystal display panels, microelectromechanical systems displays (e.g., two-dimensional mirror arrays or scanning mirror display devices), display panels having pixel arrays formed from crystalline semiconductor light-emitting diode dies (sometimes referred to as microLEDs), and/or other display devices.

Sensors 16 in input-output devices 24 may include force sensors (e.g., strain gauges, capacitive force sensors, resistive force sensors, etc.), audio sensors such as microphones, touch and/or proximity sensors such as capacitive sensors such as a touch sensor that forms a button, trackpad, or other input device), and other sensors. If desired, sensors 16 may include optical sensors such as optical sensors that emit and detect light, ultrasonic sensors, optical touch sensors, optical proximity sensors, and/or other touch sensors and/or proximity sensors, monochromatic and color ambient light sensors, image sensors, fingerprint sensors, iris scanning sensors, retinal scanning sensors, and other biometric sensors, temperature sensors, sensors for measuring three-dimensional non-contact gestures (“air gestures”), pressure sensors, sensors for detecting position, orientation, and/or motion (e.g., accelerometers, magnetic sensors such as compass sensors, gyroscopes, and/or inertial measurement units that contain some or all of these sensors), health sensors such as blood oxygen sensors, heart rate sensors, blood flow sensors, and/or other health sensors, radio-frequency sensors, depth sensors (e.g., structured light sensors and/or depth sensors based on stereo imaging devices that capture three-dimensional images), optical sensors such as self-mixing sensors and light detection and ranging (lidar) sensors that gather time-of-flight measurements, humidity sensors, moisture sensors, gaze tracking sensors, electromyography sensors to sense muscle activation, facial sensors, and/or other sensors. In some arrangements, device 10 may use sensors 16 and/or other input-output devices to gather user input. For example, buttons may be used to gather button press input, touch sensors overlapping displays can be used for gathering user touch screen input, touch pads may be used in gathering touch input, microphones may be used for gathering audio input, accelerometers may be used in monitoring when a finger contacts an input surface and may therefore be used to gather finger press input, etc.

If desired, electronic device 10 may include additional components (see, e.g., other devices 18 in input-output devices 24). The additional components may include haptic output devices, actuators for moving movable housing structures, audio output devices such as speakers, light-emitting diodes for status indicators, light sources such as light-emitting diodes that illuminate portions of a housing and/or display structure, other optical output devices, and/or other circuitry for gathering input and/or providing output. Device 10 may also include a battery or other energy storage device, connector ports for supporting wired communication with ancillary equipment and for receiving wired power, and other circuitry.

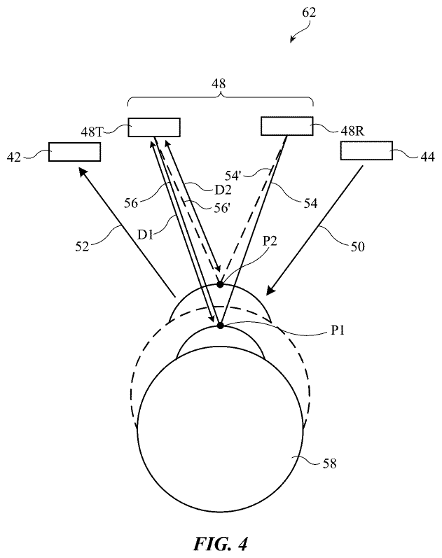

FIG. 4 is a top view of illustrative gaze tracking circuitry 62. As shown in FIG. 4, gaze tracking circuitry 62 may include one or more cameras such as camera 42, one or more light sources such as light source 44, and one or more range finders such as range finder 48. Range finder 48 may be configured to measure the distance to eye 58 (e.g., the distance to the point of specular reflection on the cornea, sometimes referred to as eye distance), sometimes referred to as eye distance). As the user's gaze moves around (e.g., from position P1 to position P2), the distance to eye 58 may change. At position P1, range finder 48 may measure a distance D1 to eye 58. At position P2, range finder 48 may measure a distance D2 to eye 58.

Range finder 48 may include one or more transmitters such as transmitter 48T and one or more receivers such as receiver 48R. Transmitter 48T may be configured to emit signal 56 toward the user's eye 58. Signal 56 may reflect off of eye 58 and reflected signal 54 may be detected by receiver 48R. If desired, range finder 48 may include more than one transmitter 48T and/or more than one receiver 48R. For example, range finder 48 may include a second transmitter 48T and a second receiver 48R for redundancy. Arrangements in which range finder 48 includes three or more transmitters 48T and/or three or more receivers 48R may also be used.

In some arrangements, one device may serve as both transmitter 48T and receiver 48R. For example, a flexible membrane in a transducer may be used to detect ultrasonic signals (when serving as receiver 48R) and may also be used to emit ultrasonic signals (when serving as transmitter 48T). As another example, a self-mixing interferometer may also serve as both transmitter 48T and receiver 48R.

Range finder 48 may be any suitable sensor configured to measure distance. In arrangements where range finder 48 is an optical sensor (e.g., an optical sensor that gathers time-of-flight measurements, a self-mixing sensor, a light detection and ranging (lidar) sensor, a structured light sensor, a phased-based optical coherence tomography sensor, and/or a depth sensor based on a stereo imaging device that captures three-dimensional images, etc.), emitted signal 56 and reflected signal 54 may be optical signals. When range finder 48 is formed from a phased-based sensor such as an optical sensor based on optical coherence tomography, range finder 48 may be configured to achieve smaller resolvable time intervals than time-of-flight based sensors. In arrangements where range finder 48 is an ultrasonic sensor (e.g., one or more capacitive micromachined ultrasonic transducers, piezoelectric micromachined transducers, and/or other suitable ultrasonic transducers for emitting and/or detecting acoustic signals), emitted signal 56 and reflected signal 54 may be ultrasonic signals.

If desired, transmitter 48T may be co-located with receiver 48R. In other arrangements, transmitter 48T and receiver 48R may be mounted in different locations. Camera 42 and light source 44 may be co-located with one another or may be mounted in different locations. One or both of transmitter 48T and receiver 48R may be co-located with camera 42 and/or light source 44, or transmitter 48T and receiver 48R may be mounted separately from camera 42 and/or light source 44.

During operation, light source 44 may be used to emit light 50 towards eye 58. Light 50 may reflect off of eye 58 and reflected light 52 may be detected by camera 42. Emitted light 50 may create a glint on eye 58. Camera 42 may capture images of eye 58 including the glint created by light 50. Based on the captured images, gaze tracking circuitry 62 may determine the location of the glint and the location of the user's pupil. In some arrangements, there may be multiple light sources 44 that produce multiple glints on the user's eye. If there are a sufficient number of glints produced on eye 58, gaze control circuitry 62 can determine the shape of the user's eye (e.g., the user's cornea), which in turn can be used to determine gaze direction (e.g., without requiring range finder 48).

In some arrangements, such as when fewer light sources 44 are desired, or when a sufficient number of glints cannot be captured due to the shape of a particular user's eye, gaze control circuitry 62 may combine glint detection with distance sensing to determine gaze direction. In this type of scenario, a single light source 44 may produce a single glint on eye 58, and camera 42 may capture images of eye 58 including the single glint. Based on the captured images, gaze tracking control circuitry 62 may determine a position of the glint and a position of the pupil. Because the eye is mostly spherical (e.g., to first order), the glint on eye 58 will remain mostly in the same place as the eye moves around, but the position of the pupil relative to the glint will change as the gaze direction changes. In particular, as the eyeball moves around to different gaze directions (e.g., from position to P1 to position P2), the position of the pupil relative to the glint will change by a scaling factor that depends on the distance to the eyeball. By using range finder 48 to determine a distance to the eyeball at positions P1 and P2, gaze tracking circuitry 62 can determine this scaling factor and can therefore map the pupil and glint positions at P1 to a first gaze direction (e.g., based on distance D1) and the pupil and glint positions at P2 to a second gaze direction (e.g., based on distance D2).

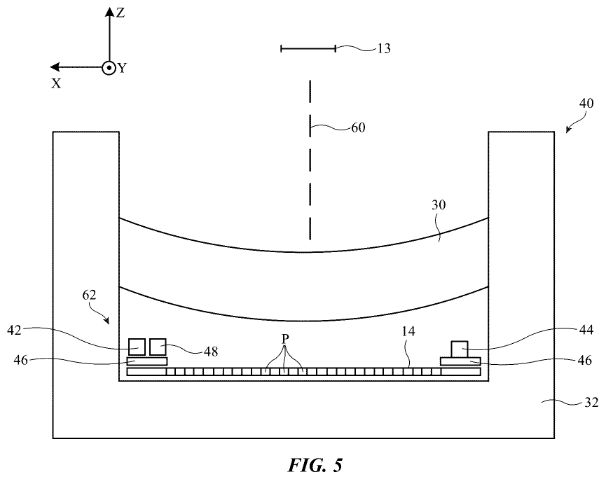

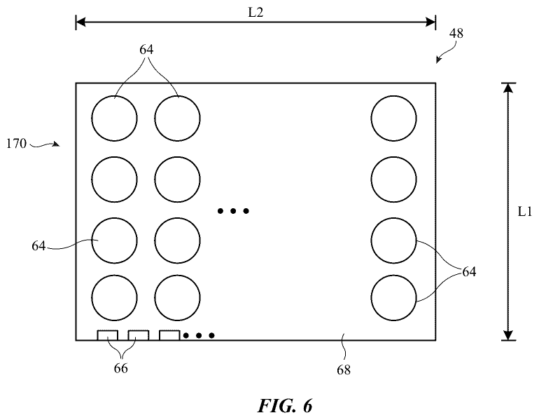

Device 10 may include gaze tracking circuitry 62 for each eye 58 (e.g., a left eye 58 and a right eye 58), or device 10 may include gaze tracking circuitry 62 for a single eye 58. FIG. 5 is a side view of a portion of device 10 showing an illustrative example of how gaze tracking circuitry 62 may be implemented in a device that includes a head-mounted display.

In the example of FIG. 5, gaze tracking circuitry 62 is mounted in optical module 40 of device 10. This is merely illustrative. If desired, gaze tracking circuitry 62 may be mounted in other locations of device 10. Optical module 40 may have lens barrel 32. Lens 30 may be used to provide an image from pixels P of display 14 to eye box 13 along optical axis 60. To provide eye illumination that illuminates an eye that is located in eye box 13, module 40 may contain one or more light sources (e.g., lasers, light-emitting diodes, lamps, etc.) such as one or more light-emitting diodes 44. One or more cameras 42 may be included in each optical module 40 to monitor eye box 13. Camera 42 may capture images of the user's eye while the user's eye is located in eye box 13.

Light-emitting diodes 44 may emit light at one or more wavelengths of interest (e.g., visible light wavelengths and/or infrared light wavelengths, etc.) and camera 42 may be sensitive at these wavelengths (e.g., visible light wavelengths and/or infrared light wavelengths, etc.). In an illustrative configuration, light-emitting diodes 44 emit infrared light. Infrared light may be used to illuminate the user's eye in eye box 13 while being unnoticeable (or nearly unnoticeable) to the user (e.g., because human vision is not generally sensitive to infrared light except when the infrared light has an infrared wavelength near the edge of the visible light spectrum, which extends from 380 to 740 nm).

Electronic components in module 40 such as display 14, camera 42, light-emitting diodes 44, and range finder 48 may be coupled to flexible printed circuits or other substrates containing metal traces. The metal traces may form interconnect paths that carry power signals, data signals, and control signals. As shown in FIG. 5, for example, light-emitting diodes 44 may be mounted on a ring-shaped substrate such as flexible printed circuit 46. Printed circuit 46 and light-emitting diodes 44 may extend around some or all of the inner periphery of lens barrel 32 (and therefore around some or all of the outer periphery of display 14).

During operation, light from light-emitting diodes 44 that are mounted along the edge of display 14 may travel to eye box 13 through lens 30. Light-emitting diodes 44 are generally out of the user's field of view or nearly out of the user's field of view as the user is viewing images presented by the array of pixels P on display 14. Some of light-emitting diodes 44 may create glints (e.g., reflections) off of the surface of the user's eye in eye box 13. These reflections can be captured by camera 42. Device 10 can process glint information obtained by cameras 24 to track the user's gaze. For example, control circuitry 20 can analyze the positions of the glints to determine the shape of the user's eye (e.g., the user's cornea). From this information, control circuitry 20 can determine the direction of the user's gaze.

In addition to serving as glint light sources (e.g., light sources that produce glint illumination that is detected as discrete eye glints by camera 42), light from light-emitting diodes 44 may serve as blanket eye illumination, if desired. In particular, light from light-emitting diodes 44 may illuminate portions of each of the user's eyes such as the user's iris and the user's pupil.

During operation, camera 42 can capture an image of the user's pupil as the pupil is being illuminated by light from diodes 44. The user's pupil will have a shape (e.g., an oval shape) that varies depending on the orientation of the user's eye to camera 42. If, as an example, the eye is aligned with camera 42, the pupil will appear circular or nearly circular, whereas if the eye is angled away from camera 42, the pupil will have higher eccentricity. By analyzing the shape of the pupil, control circuitry 20 can determine the direction of the user's gaze.

It may also be desirable for camera 42 to capture other eye images such as images of the iris of the user's eye. Iris patterns are user-specific, so iris images may be used to authenticate users in device 10 (e.g., to log the user into a user account, to substitute for a username and/or password, or to otherwise serve as a biometric credential for device 10).

Pupil illumination and the illumination for the glints can be produced by light-emitting diodes 44 at the same wavelength or at different wavelengths. For example, pupil and glint illumination can be provided by light-emitting diodes 44 at a wavelength of 940 nm, 800-1000 nm, at least 800 nm, at least 850 nm, at least 900 nm, at least 950 nm, less than 950 nm, or other suitable wavelength. Configurations in which the wavelength of the glint and pupil illumination is sufficiently long to be invisible to most or all users may help allow glint and pupil measurements and/or other gaze tracking measurements to be taken continuously during operation of device 10, without potentially distracting users. Iris illumination may be provided by light-emitting diodes 44 at the same wavelength and/or a different wavelength than the glint illumination and the pupil illumination. To obtain desired image contrast when gathering iris information, it may be desirable for iris illumination to be provided at a shorter wavelength than the pupil and glint illumination (e.g., at a visible light wavelength and/or at a shorter infrared wavelength than used by diodes 44 when providing gaze tracking illumination). Camera 42 may include a single image sensor that captures pupil image data, glint image data, and iris image data, and/or multiple cameras may be provided each of which captures image data at a different wavelength (or band of wavelengths).

It can be challenging to capture a sufficient number of glints on every type of eye. If desired, gaze tracking circuitry 62 may be a hybrid gaze tracker that combines glint detection with range finding (distance sensing) to determine a user's gaze direction using fewer glints (e.g., using as few as one glint on the eye). As discussed in connection with FIG. 4, determining a distance to the user's eye using range finder 48 allows gaze tracking circuitry 62 to determine gaze direction using only a single light-emitting diode 44 and a single glint on the eye captured by camera 42. Additionally, since it can be assumed that the gaze direction is unchanged if the distance to the eye is unchanged, gaze tracking circuitry 62 can selectively activate camera 42 and light source 44 only when needed, if desired. For example, after determining gaze direction using light source 44, camera 42, and range finder 48, gaze detection circuitry 62 may turn off light source 44 and camera 42 (or may otherwise switch light source 44 and camera 42 to a low-power state) while continuing to gather distance measurements using range finder 48. If there is no change detected in the distance to the eye, gaze detection circuitry 62 may assume that the previously determined gaze direction is unchanged. If there is a change in eye distance detected by range finder 48, light source 44 and camera 42 may be activated (e.g., turned on) to illuminate the eye with light source 44 while capturing images of the eye using camera 42. Based on the new pupil and glint positions and the new distance to the eye measured by range finder 48, gaze tracking circuitry 62 may determine a new gaze direction of the user.

FIG. 6 is a top view of an illustrative range finder 48 that is formed using ultrasonic transducers. As shown in FIG. 6, range finder 48 may include one or more arrays 170 of ultrasonic transducers such as ultrasonic transducers 64. Each array 170 may include multiple ultrasonic transducers 64 on a substrate such as substrate 68. Ultrasonic transducers 64 within each array 170 may have different center frequencies such that, when used together, the array can collectively achieve the positioning accuracy of a wideband ultrasonic transducer without experiencing the reduction in quality factor that would normally result from a single broadband ultrasonic transducer. Arrays 170 may be mounted in housing 12 of device 10 and may be distributed at different locations around the eye of the user (e.g., around the left and right eyes of the user). There may be any suitable number of arrays 170 in gaze tracking circuitry 62 (e.g., one, two, three, four, five, six, ten, fifteen, twenty, more than twenty, less than twenty, etc.), and each array 170 may include any suitable number of transducers 64 (e.g., five, eight, ten, fifteen, twenty, fifty, one hundred, two hundred, less than two hundred, more than two hundred, etc.).

One or more of arrays 170 may be used to form transmitter 48T of FIG. 4 and may emit ultrasonic signals (e.g., emitted signals 56 of FIG. 4). The ultrasonic signals 56 may reflect off of a user's eye 58 (e.g., may reflect off of the cornea of the user's eye). One or more of arrays 170 may form receiver 48R of FIG. 4 and may be used to detect reflected ultrasonic signals 54 after the signals reflect off of the user's cornea. Using time-of-flight measurement techniques, control circuitry 20 may be used to determine the time that it takes for the emitted signal 56 to reflect back from eye 58, which may in turn be used to determine the distance to eye 58 (e.g., the distance to the point of specular reflection on the cornea). If desired, the same array 170 may be used to emit and detect the ultrasonic signals. Arrangements in which multiple arrays 170 emit ultrasonic signals and/or where multiple arrays 170 detect ultrasonic signals may also be used.

If desired, one or more of arrays 170 may be a phased transducer array. In a phased ultrasonic transducer array, beam steering techniques may be used in which ultrasonic signal phase and/or magnitude for each transducer in array 170 are adjusted to perform beam steering. Beam steering may be used to “illuminate” a particular area of interest with ultrasonic signals and/or to illuminate other arrays 170 with a calibration signal. Beam steering may also be used to avoid illuminating certain areas with ultrasonic signals (e.g., to avoid directly illuminating other arrays 170 and/or to avoid illuminating certain parts of the user's face). For example, a phased ultrasonic transducer array 170 may be configured to emit a concentrated beam of ultrasonic signals that strikes the cornea but does not strike the user's eye brow. This type of beam steering arrangement may help improve gaze tracking accuracy by avoiding detecting significant reflections from surfaces around the user's eye.

The use of time-of-flight based measurement techniques is merely illustrative. If desired, other time-based, amplitude-based, and/or phase-based measurement schemes such as time-difference-of-arrival measurement techniques, angle-of-arrival measurement techniques, triangulation methods, and/or other suitable measurement techniques may be used to determine a location of the user's eye using ultrasonic sensor arrays 170.

As shown in FIG. 6, transducer array 170 may include multiple transducers 64 on substrate 68. Array 170 may include transducers 64 with different center frequencies. The center frequency of an ultrasonic transducer may refer to the frequency at the center of the frequency range of which the transducer is capable of operating. For example, array 170 may include transducers with two, three, four, five, six, seven, ten, more than ten, or less than ten center frequencies. The center frequencies of transducers 64 may, for example, be between 750 kHz and 1.25 MHz, between 500 kHz and 1.25 MHz, between 700 kHz and 1 MHz, between 800 kHz and 1.2 MHz, between 900 kHz and 1.1 MHz, between 750 kHz and 1.4 MHz, or between any other suitable frequency range. There may be any suitable number (e.g., one, two, three, four, five, more than five, less than five) of transducers 64 in array 170 for a given center frequency. If desired, each transducer 64 may have one or more natural oscillation frequencies that are used for the excitation or detection of ultrasound waves.

The bandwidth of each individual transducer 64 may be smaller than the collective bandwidth spanned by all of the transducers 64 in array 170. The center frequencies of individual transducers 64 may be selected so that the collective bandwidth of the entire array 170 spans some or all of the desired frequency range (e.g., from 750 kHz to 1.25 MHZ, from 700 kHz to 1 MHz, from 800 kHz to 1.2 MHz, from 900 kHz to 1.1 MHz, from 750 kHz to 1.4 MHz, or any other suitable frequency range). The desired frequency range may depend on the range of distances to be measured. For example, to measure distances to objects that are within a few centimeters (such as a user's eye 58), array 170 may span a frequency range of 750 kHz to 1.25 MHz (as an example).

Substrate 68 may have any suitable dimensions. For example, lateral dimensions L1 and L2 of substrate 68 may be between 2 mm and 2.5 mm, between 1 mm and 1.5 mm, between 1 mm and 3 mm, between 2 mm and 4 mm, and/or other suitable length. Dimensions L1 and L2 may be equal (so that substrate 68 has a square footprint) or unequal (so that substrate 68 has a rectangular footprint), or the footprint of substrate 68 may have other shapes (e.g., circular, oval, round, triangular, etc.).

Transducers 64 may be arranged in an evenly spaced grid of rows and columns on substrate 68 or may be arranged with any other suitable pattern (e.g., unevenly spaced clusters, a random pattern, a non-grid pattern, etc.). The example of FIG. 6 in which transducers 64 have a circular shape is merely illustrative. If desired, transducers 64 may be square, rectangular, oval, round, or any other suitable shape.

In some arrangements, it may be desirable to maximize the amount of space between transducers 64 that share the same center frequency. Maximizing the spacing between commonly configured transducers 64 in array 170 may increase the accuracy of distance measurements made with array 170. Different rules regarding placement of the different subsets of transducers 64 on substrate 68 may be implemented to achieve the desired performance from array 170. As an example, the convex hull of a given set of transducers 64 that share the same center frequency may cover at least 50% of array 170, may cover at least 80% of array 170, or may cover other suitable portions of array 170. As another example, most pairs of transducers 64 that share the same center frequency may be separated by a transducer 64 of a different center frequency. These examples are merely illustrative. In general, transducers 64 may be placed in any suitable arrangement on substrate 68.

Operation of transducers 64 may be controlled by control circuitry 20. Substrate 68 may include interconnects 66 for conveying signals between transducers 64 and control circuitry 20. For example, interconnects 66 may be used to convey driving signals from control circuitry 20 to transducers 64 and to convey sensor signals (e.g., sensor signals associated with ultrasonic waves that are detected by transducers 64) from transducers 64 to control circuitry 20.

If desired, transducers 64 in array 170 may be independently controlled from one another. For example, the frequency, phase, and pulse shape of the driving signal for a given transducer 64 may be different from other transducers 64 in array 170. Each individual transducer 64 in array 170 may be independently controlled with different driving signals, or there may be subsets of transducers 64 (e.g., a subset that share the same center frequency or other suitable subset) that are controlled with the same drive signals but that are independently controlled from other subsets of transducers 64. This is merely illustrative, however. If desired, transducers 64 may not be independently controlled and/or may be controlled with any other suitable driving scheme.

In some arrangements, transducers 64 may be driven by off-chip control circuitry. In this type of arrangement, interconnects 66 may include leads, contact pads, solder and/or other conductive elements for conveying signals between array 170 control circuitry 20 that is separate from array 170. In other arrangements, substrate 68 may be a multilayer substrate in which transducers 64 are stacked with a control circuitry layer (e.g., an application-specific integrated circuit layer) that includes control circuitry 20. With this type of integrated control circuitry, interconnects 66 may include metal vias or and/or other conductive elements for conveying signals between transducers 64 and control circuitry 20 that is located in a different layer of substrate 68. These examples are merely illustrative. If desired, interconnects 66 may include metal vias for conveying signals between different layers of substrate 68 and may also include contact pads for conveying signals between array 170 and external circuitry.

The center frequency of a piezoelectric transducer is determined at least in part by the dimensions of the cavity of the transducer. In some arrangements, transducers 64 may be provided with different center frequencies by using cavities with different dimensions (e.g., different depths, different diameters, etc.) and/or by using different surface features within the cavities to create the desired acoustic reflection phase at the center frequency. If desired, other structures may be used to produce an array of transducers with different center frequencies. For example, instead of varying the cavity depth, transducers 64 may have uniform cavity depth (e.g., may all have a relatively short cavity depth) but with different lateral cavity dimensions (e.g., different diameters, different lengths and widths, etc.). Arrangements in which both cavity depth and the lateral dimensions of the cavity are varied may also be used. In general, any suitable technique for producing transducers with different center frequencies may be used. In addition to or instead of adjusting cavity size to achieve the desired center frequency, the mechanical resonance of the membrane of transducer 64 may be adjusted to tune the resonance frequency of transducer 64. In particular, one or more openings may be formed in the membrane to adjust the spring constant of the membrane and thereby adjust its resonance frequency.

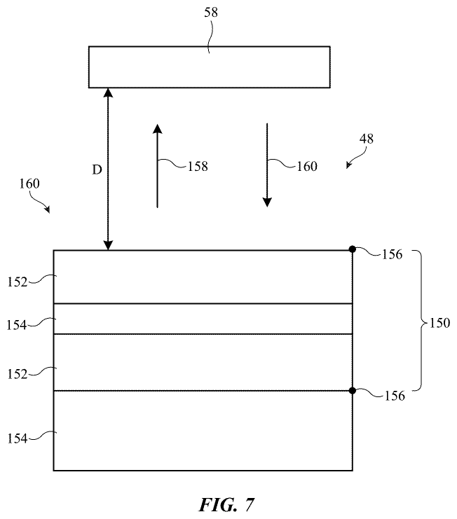

An illustrative optical self-mixing sensor is shown in FIG. 7. Self-mixing sensor 160, which may sometimes be referred to as an optical self-mixing position sensor or self-mixing orientation sensor may be used to measure distance and therefore determine the relative position between the sensor and a target structure. In some configurations, angular orientation may be measured using one or more self-mixing sensors. For example, angular tilt may be measured by measuring two or more distances. Tilt about one axis may, as an example, be measured using a pair of distance measurements made at different respective locations on a component, whereas tilt about two axes may be measured using three such distance measurements. Arrangements in which self-mixing sensors are referred to as measuring distance, displacement, or position may sometimes be described herein as an example. In general, position, angular orientation, changes in position and/or orientation, and/or other self-mixing sensor measurements may be directly gathered and/or may be derived from the measurements of distance from self-mixing sensors.

In some arrangements, range finder 48 may include a self-mixing sensor such as a self-mixing optical interferometer. FIG. 7 is a side view of an illustrative self-mixing interferometer such as self-mixing interferometer 160. As shown in FIG. 7, self-mixing sensor 160 may be used to measure the separation (distance D) between sensor 160 and eye 58.

Self-mixing interferometer 160 may include a laser such as vertical cavity surface emitting laser 150 (e.g., self-mixing proximity sensor 160 may be a coherent self-mixing sensor having a diode laser or other coherent or partially coherent source of light or other electromagnetic radiation). Laser 150 may have thin-film interference filter mirrors 152 (sometimes referred to as Bragg reflectors) each of which is formed from a stack of thin-film layers of alternating index of refraction. Active region 154 may be formed between mirrors 152. The lower mirror 152 in laser 150 may have a nominal reflectivity of less than 100% to allow some of the light of laser 150 to reach overlapped photodiode 154 or, in configurations in which photodiode 154 is located elsewhere in sensor 160 (e.g., laterally adjacent to laser 150), the lower mirror 152 may have a nominal reflectivity of 100%. The upper mirror 152 in laser 150 may have a slightly lower reflectivity, so that laser 150 emits light 158 towards eye 58. Laser 150 may be controlled by applying a drive signal to terminals 156 using control circuitry 20 (e.g., a drive circuit in circuitry 20). Sensing circuitry (e.g., photodiode 154 and/or associated sensing circuitry in circuitry 20) can measure the light output of laser 150 (as an example).

Emitted light 158 may have an infrared wavelength of 850-1200 nm, 800 nm to 1100 nm, 920-960 nm, at least 800 nm, at least 900 nm, at least 1000 nm, less than 1200 nm, less than 1100 nm, less than 1000 nm, or less than 900 nm, or other suitable wavelength (e.g., a visible wavelength, an ultraviolet wavelength, an infrared wavelength, a near-infrared wavelength, etc.). When emitted light 158 illuminates eye 58, some of emitted light 158 will be reflected backwards towards sensor 150 as reflected light 160 (e.g., light that is specularly reflected from eye 58 and/or that is backscattered from a matte surface in eye 58).

Sensor 160 of FIG. 7 includes a light sensitive element (e.g., a light detector such as photodiode 154). Photodiode 154 in the example of FIG. 7 is located under laser 150, but configurations in which photodiode 154 is adjacent to laser 150, is located on a separate substrate than laser 150, is located above active area 154 of laser 150, and/or has other configurations may be used, if desired. The terminals of photodiode 154 may be coupled to sensing circuitry in control circuitry 20. This circuitry gathers photodiode output signals that are produced in response to reception of reflected light (specularly reflected and/or backscattered portions of emitted light 158) such as reflected light 160. In addition to using a photodiode, self-mixing can be detected using laser junction voltage measurements (e.g., if the laser is driven at a constant bias current) or laser bias current (e.g., if the laser is driven at a constant voltage).

Some of light 160 that is reflected or backscattered from eye 58 as reflected light 160 reenters the laser cavity of laser 150 and mixes with the light in the laser cavity, perturbing the electric field coherently and causing a perturbation to the carrier density in laser 150. These perturbations in laser 150 cause coherent self-mixing fluctuations in the power of emitted light 158 and associated operating characteristics of laser 150 such as laser junction voltage and/or laser bias current. These fluctuations may be monitored. For example, the fluctuations in the power of light 158 may be monitored using photodiode 154. In the example of FIG. 7, photodiode 154 is an integrated monolithic photodiode that is formed under laser 150, but other configurations may be used, if desired.

Control circuitry 20 is configured to supply drive current for laser 150 and includes circuitry for sensing the response of photodiode 154. Sensed photodiode output may include measurements of diode current and/or voltage. A modulation scheme may be used for driving laser 150 for the purpose of inducing a wavelength modulation and a photodiode output processing scheme (using measurements of photodiode current, junction voltage, bias current, etc.) may be used in processing the measured self-mixing fluctuations in output power to allow control circuitry 20 to determine the distance D between sensor 160 and eye 58 in accordance with the principles of self-mixing interferometry.

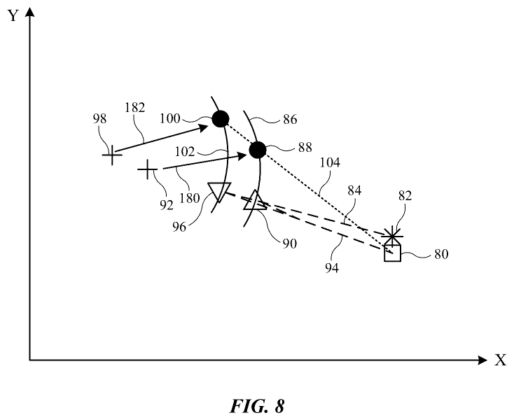

The example of FIGS. 7 and 8 are merely illustrative examples of range finders 48 that may be included in gaze tracking circuitry 62 of device 10. If desired, range finder 48 may include additional or different types of distance sensors.

FIG. 8 is a graph showing an illustrative quantitative model for determining gaze direction using camera 42, light-emitting diode 44, and range finder 48. While FIG. 8 illustrates a two-dimensional model, the two-dimensional model may be generalized to three dimensions. In gaze tracking circuitry 62, camera 42, light source 44, and the corneal center of eye 58 (FIG. 4) define three points in space and therefore define a plane. The glint produced on eye 58 by light source 44 may also be contained within this same plane (due to symmetry). In this model, it may be assumed that the cornea is locally spherical around the regions where the glint is produced and from which distance is measured. Additionally, it may be assumed that camera 42 can find a bearing to the apex of the cornea (e.g., the point directly above the pupil) and a bearing to the glint.

The model of FIG. 8 shows how geometry can be used to predict a ground truth. By adding in errors in range (distance) data and/or cornea radius and performing gaze reconstruction using this imperfect data, the sensitivity of the model to errors can be determined. In a forward model, a ground truth optical axis (sometimes referred to as a ground truth gaze direction) may be used to determine pupil location 88 and corneal center 92. From corneal center 92, corneal surface 86 may be determined. The location on cornea 86 that minimizes the round trip from camera 42 at location 80 to light source 44 at location 82 yields a ground truth time-of-flight path measurement (see path 84 of FIG. 8). This location 90 on cornea 86 is where the glint will appear. The ground truth optical axis is indicated by line 180 that joins cornea center 92 with pupil location 88.

To determine the model's sensitivity to errors, a range measurement error may be assumed. For example, it may be assumed that camera 42 has a negligible error in glint bearing, that range finder 48 detects an inaccurate eye distance, or that there is noise in the glint image captured by camera 42 that adds additional length on the ray between camera 42 and the real glint. This may result in a modeled glint at location 96 instead of location 90. Based on the modeled glint at location 96, cornea surface 102 and estimated cornea center 98 can be determined (e.g., based on a known radius of curvature of the cornea). The bearing 104 of the pupil from camera 42 may be projected until it intersects with modeled cornea 102 at modeled pupil location 100. Line 182 joining modeled cornea center 98 and modeled pupil location 100 corresponds to the modeled gaze direction of the user. Assuming that the time-of-flight error is about 0.5 mm, the model may still be able to predict a sufficiently accurate gaze direction of the user. In general, the gaze direction error may scale linearly with range measurement error.

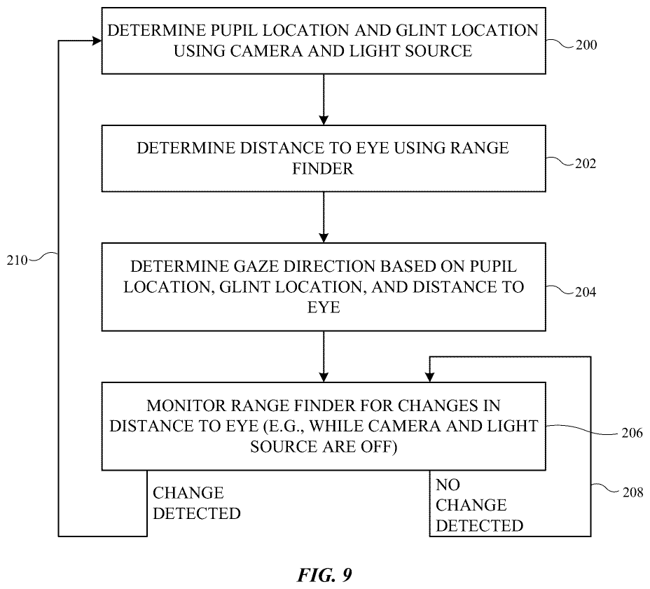

FIG. 9 is a flow chart of illustrative steps involved in determining a user's gaze direction using gaze tracking circuitry 62.

During the operations of block 200, light source 44 may be used to produce a glint on the user's eye 58 (FIG. 4). Camera 42 may capture images of the eye including the glint. Based on the captured images (and based on other known data such as a radius of corneal curvature), control circuitry 20 may determine the location of the user's pupil and the location of the glint.

During the operations of block 202, range finder 48 may be used to determine a distance to the eye. This may include emitting optical signals, ultrasonic signals, and/or other suitable signals towards the eye using a transmitter (e.g., transmitter 48T of FIG. 4) and detecting the reflected signals using a receiver (e.g., receiver 48R of FIG. 4). Using time-of-flight measurement techniques or other suitable measurement techniques, the distance to the eye can be determined.

During the operations of block 204, control circuitry 20 may determine the gaze direction of the user based on the measured pupil location, the measured glint location, and the measured distance to the eye.

It may be assumed that the gaze direction is unchanged if the distance to the eye is also unchanged. If desired, camera 42 and light source 44 may be temporarily placed in a low-power state (e.g., an off state, a sleep state, or other low-power state) after gaze direction is determined to save power, while range finder 48 may be used to monitor for changes in distance to the eye. During the operations of block 206, for example, camera 42 and light source 44 may be placed in a low-power state while range finder 48 continuously or periodically measures the distance to the eye. If no change in distance is detected, processing may loop back to step 206 (see line 208 of FIG. 9) and control circuitry 20 may continue monitoring for changes in distance to the eye using range finder 48. If a change in distance is detected, camera 42 and light source 44 may be turned on (e.g., switched from a low-power state to a powered-on state) and processing may loop back to step 200 (see line 210 of FIG. 9). This allows camera 42 and light source 44 to be turned on only when a new gaze direction needs to be measured, if desired.

This is, however, merely illustrative. If desired, camera 42 and light source 44 may remain powered on and may be used in conjunction with range finder 48 to determine gaze direction.

As described above, one aspect of the present technology is the gathering and use of information such as information from input-output devices. The present disclosure contemplates that in some instances, data may be gathered that includes personal information data that uniquely identifies or can be used to contact or locate a specific person. Such personal information data can include demographic data, location-based data, telephone numbers, email addresses, social media information, home addresses, data or records relating to a user's health or level of fitness (e.g., vital signs measurements, medication information, exercise information), date of birth, username, password, biometric information, or any other identifying or personal information.

The present disclosure recognizes that the use of such personal information, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the United States, collection of or access to certain health data may be governed by federal and/or state laws, such as the Health Insurance Portability and Accountability Act (HIPAA), whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to provide certain types of user data. In yet another example, users can select to limit the length of time user-specific data is maintained. In addition to providing “opt in” and “opt out” options, the present disclosure contemplates providing notifications relating to the access or use of personal information. For instance, a user may be notified upon downloading an application (“app”) that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of information that may include personal information data to implement one or more various disclosed embodiments, the present disclosure also contemplates that the various embodiments can also be implemented without the need for accessing personal information data. That is, the various embodiments of the present technology are not rendered inoperable due to the lack of all or a portion of such personal information data.

Physical environment: A physical environment refers to a physical world that people can sense and/or interact with without aid of electronic systems. Physical environments, such as a physical park, include physical articles, such as physical trees, physical buildings, and physical people. People can directly sense and/or interact with the physical environment, such as through sight, touch, hearing, taste, and smell.

Computer-generated reality: in contrast, a computer-generated reality (CGR) environment refers to a wholly or partially simulated environment that people sense and/or interact with via an electronic system. In CGR, a subset of a person's physical motions, or representations thereof, are tracked, and, in response, one or more characteristics of one or more virtual objects simulated in the CGR environment are adjusted in a manner that comports with at least one law of physics. For example, a CGR system may detect a person's head turning and, in response, adjust graphical content and an acoustic field presented to the person in a manner similar to how such views and sounds would change in a physical environment. In some situations (e.g., for accessibility reasons), adjustments to characteristic(s) of virtual object(s) in a CGR environment may be made in response to representations of physical motions (e.g., vocal commands). A person may sense and/or interact with a CGR object using any one of their senses, including sight, sound, touch, taste, and smell. For example, a person may sense and/or interact with audio objects that create 3D or spatial audio environment that provides the perception of point audio sources in 3D space. In another example, audio objects may enable audio transparency, which selectively incorporates ambient sounds from the physical environment with or without computer-generated audio. In some CGR environments, a person may sense and/or interact only with audio objects. Examples of CGR include virtual reality and mixed reality.

Virtual reality: A virtual reality (VR) environment refers to a simulated environment that is designed to be based entirely on computer-generated sensory inputs for one or more senses. A VR environment comprises a plurality of virtual objects with which a person may sense and/or interact. For example, computer-generated imagery of trees, buildings, and avatars representing people are examples of virtual objects. A person may sense and/or interact with virtual objects in the VR environment through a simulation of the person's presence within the computer-generated environment, and/or through a simulation of a subset of the person's physical movements within the computer-generated environment.

Mixed reality: In contrast to a VR environment, which is designed to be based entirely on computer-generated sensory inputs, a mixed reality (MR) environment refers to a simulated environment that is designed to incorporate sensory inputs from the physical environment, or a representation thereof, in addition to including computer-generated sensory inputs (e.g., virtual objects). On a virtuality continuum, a mixed reality environment is anywhere between, but not including, a wholly physical environment at one end and virtual reality environment at the other end. In some MR environments, computer-generated sensory inputs may respond to changes in sensory inputs from the physical environment. Also, some electronic systems for presenting an MR environment may track location and/or orientation with respect to the physical environment to enable virtual objects to interact with real objects (that is, physical articles from the physical environment or representations thereof). For example, a system may account for movements so that a virtual tree appears stationery with respect to the physical ground. Examples of mixed realities include augmented reality and augmented virtuality. Augmented reality: an augmented reality (AR) environment refers to a simulated environment in which one or more virtual objects are superimposed over a physical environment, or a representation thereof. For example, an electronic system for presenting an AR environment may have a transparent or translucent display through which a person may directly view the physical environment. The system may be configured to present virtual objects on the transparent or translucent display, so that a person, using the system, perceives the virtual objects superimposed over the physical environment. Alternatively, a system may have an opaque display and one or more imaging sensors that capture images or video of the physical environment, which are representations of the physical environment. The system composites the images or video with virtual objects, and presents the composition on the opaque display. A person, using the system, indirectly views the physical environment by way of the images or video of the physical environment, and perceives the virtual objects superimposed over the physical environment. As used herein, a video of the physical environment shown on an opaque display is called “pass-through video,” meaning a system uses one or more image sensor(s) to capture images of the physical environment, and uses those images in presenting the AR environment on the opaque display. Further alternatively, a system may have a projection system that projects virtual objects into the physical environment, for example, as a hologram or on a physical surface, so that a person, using the system, perceives the virtual objects superimposed over the physical environment. An augmented reality environment also refers to a simulated environment in which a representation of a physical environment is transformed by computer-generated sensory information. For example, in providing pass-through video, a system may transform one or more sensor images to impose a select perspective (e.g., viewpoint) different than the perspective captured by the imaging sensors. As another example, a representation of a physical environment may be transformed by graphically modifying (e.g., enlarging) portions thereof, such that the modified portion may be representative but not photorealistic versions of the originally captured images. As a further example, a representation of a physical environment may be transformed by graphically eliminating or obfuscating portions thereof. Augmented virtuality: an augmented virtuality (AV) environment refers to a simulated environment in which a virtual or computer generated environment incorporates one or more sensory inputs from the physical environment. The sensory inputs may be representations of one or more characteristics of the physical environment. For example, an AV park may have virtual trees and virtual buildings, but people with faces photorealistically reproduced from images taken of physical people. As another example, a virtual object may adopt a shape or color of a physical article imaged by one or more imaging sensors. As a further example, a virtual object may adopt shadows consistent with the position of the sun in the physical environment.

Hardware: there are many different types of electronic systems that enable a person to sense and/or interact with various CGR environments. Examples include head mounted systems, projection-based systems, heads-up displays (HUDs), vehicle windshields having integrated display capability, windows having integrated display capability, displays formed as lenses designed to be placed on a person's eyes (e.g., similar to contact lenses), headphones/earphones, speaker arrays, input systems (e.g., wearable or handheld controllers with or without haptic feedback), smartphones, tablets, and desktop/laptop computers. A head mounted system may have one or more speaker(s) and an integrated opaque display. Alternatively, a head mounted system may be configured to accept an external opaque display (e.g., a smartphone). The head mounted system may incorporate one or more imaging sensors to capture images or video of the physical environment, and/or one or more microphones to capture audio of the physical environment. Rather than an opaque display, a head mounted system may have a transparent or translucent display. The transparent or translucent display may have a medium through which light representative of images is directed to a person's eyes. The display may utilize digital light projection, OLEDs, LEDs, μLEDs, liquid crystal on silicon, laser scanning light sources, or any combination of these technologies. The medium may be an optical waveguide, a hologram medium, an optical combiner, an optical reflector, or any combination thereof. In one embodiment, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface.

The foregoing is merely illustrative and various modifications can be made to the described embodiments. The foregoing embodiments may be implemented individually or in any combination.

What is claimed is:

1. Eyewear, comprising:
a support structure;
first and second lenses mounted to the support structure; and
gaze tracking circuitry configured to track gaze direction, wherein the gaze tracking circuitry comprises:
a light-emitting diode configured to produce an eye glint;
a camera configured to capture an image of the eye glint; and
a range finder configured to measure eye distance, wherein the gaze direction is determined based on the captured image and the measured eye distance.

2. The eyewear defined in claim 1 wherein the range finder comprises an ultrasonic range finder.

3. The eyewear defined in claim 2 wherein the ultrasonic range finder comprises an array of ultrasonic transducers on a common substrate.

4. The eyewear defined in claim 3 wherein the array of ultrasonic transducers includes at least some ultrasonic transducers with different center frequencies.

5. The eyewear defined in claim 1 wherein the range finder comprises an optical range finder.

6. The eyewear defined in claim 5 wherein the optical range finder comprises a self-mixing interferometer.

7. The eyewear defined in claim 1 wherein the first and second lenses are adjustable prescription lenses.

8. The eyewear defined in claim 1 further comprising:
first and second displays configured to present images, wherein the images are viewable respectively through the first and second lenses from first and second eye boxes.

9. The eyewear defined in claim 1 wherein the eye glint is the only eye glint in the captured image.

10. The eyewear defined in claim 1 wherein a pupil location and a glint location are determined based on the captured image and wherein the gaze direction is determined based on the pupil location, the glint location, and the eye distance.

11. Gaze tracking circuitry, comprising:
a light source configured to produce an eye glint;
a camera configured to capture an image of the eye glint, wherein a pupil location and a glint location are determined based on the captured image; and
a range finder configured to measure eye distance, wherein gaze direction is determined based on the pupil location, the glint location, and the eye distance.

12. The gaze tracking circuitry defined in claim 11 wherein the light source comprises a light-emitting diode and wherein the eye glint is the only eye glint in the captured image.

13. The gaze tracking circuitry defined in claim 11 wherein the range finder comprises an ultrasonic range finder having an array of ultrasonic transducers on a common substrate.

14. The gaze tracking circuitry defined in claim 11 wherein the range finder comprises an optical range finder.

15. The gaze tracking circuitry defined in claim 14 wherein the optical range finder comprises a self-mixing interferometer.

16. A head-mounted device, comprising:
a display configured to present images;
a lens through which the images are viewable from an eye box; and
gaze tracking circuitry configured to track gaze direction, wherein the gaze tracking circuitry comprises:
a light source and camera that are used to determine a pupil location and a glint location; and
a range finder configured to measure eye distance, wherein the gaze direction is determined based on the pupil location, the glint location, and the eye distance.

17. The head-mounted device defined in claim 16 wherein the light source comprises a light-emitting diode that produces an eye glint, wherein the camera captures an image of the eye glint, and wherein the pupil location and the glint location are determined based on the captured image.

18. The head-mounted device defined in claim 16 wherein the range finder comprises an ultrasonic range finder.

19. The head-mounted device defined in claim 16 wherein the range finder comprises an optical range finder based on optical coherence tomography.

20. The head-mounted device defined in claim 16 wherein the range finder is configured to monitor for changes in the eye distance while the light source and the camera are in a low-power state, and wherein the light source and the camera are switched from the low-power state to a powered-on state in response to a change in the measured eye distance.
